(19) United States
(12) Patent Application Publication
(10) Pub. No.: US 2010/0069884 A1
(43) Pub. Date: Mar. 18, 2010

(54) Multi-Shape Catheter Assembly

(76) Inventor: Kusai Saadeldin Aziz, Visalia, CA (US)

Correspondence Address: Ashok Tankha, 36 Greenleigh Drive, Sewell, NJ, 08080, US

(21) Appl. No.: 12/559,516

(22) Filed: Sep. 15, 2009

Related U.S. Application Data
(60) Provisional application No. 61/096,859, filed on Sep. 15, 2008.

(51) Int. Cl. A61M 25/088

(52) U.S. Cl. 604/528

(57) ABSTRACT

A catheter assembly configurable to multiple shapes is provided. The catheter assembly comprises a detachable, interchangeable and configurable outer catheter and a detachable, interchangeable and flexible inner catheter. The shape of the outer catheter is configured to facilitate guiding of the inner catheter into an inner cavity of a patient's anatomy. The inner catheter is generally coaxial with the outer catheter. The second flexible distal end of the inner catheter enables the inner catheter to engage the inner cavity of the patient's anatomy. A locking element is provided for securing the inner catheter to the outer catheter, when the second flexible distal end of the inner catheter engages the inner cavity of the patient's anatomy to allow equipment or material to be introduced into the inner cavity. The catheter assembly is configured and manipulated to fit inner cavities of multiple anatomies for enabling a medical practitioner to perform medical procedures.

### CROSS REFERENCE TO RELATED APPLICATIONS

This application claims the benefit of provisional patent application No. 61/096,859 titled “Multi shape cardiac and vascular catheter”, filed on Sep. 15, 2008 in the United States Patent and Trademark Office.

### BACKGROUND

Typically, catheters are thin, flexible tubular devices sized for insertion into body passages and cavities, for example, the vascular system, the gastrointestinal system, etc. of a patient. A catheter is typically introduced into a vein or artery and guided to selected sites in the vascular system for passing one or more devices or fluids into a body lumen or cavity accessed by the catheter. The catheters are, for example, used to diagnose conditions and diseases of the heart, the circulatory system, etc.

In order to perform a diagnostic cardiac procedure, different catheters are used to engage the origins of the main left and right coronary arteries since these arteries are of different shapes and forms due to genetic variations. Selecting the required size and shape of the catheters to suit individual cases and conditions requires time. In the case of an acute heart attack, for example, time is very crucial. Moreover, there is an individual variation in the width of the thoracic aorta which requires catheters of different sizes. Furthermore, tortuosity of the aorta can sometimes prevent some of these catheters to engage. Also, the non-engagement of a catheter arises due to variable anatomies when percutaneous coronary intervention is planned.

A catheter is also used for peripheral vascular procedures, both diagnostic and interventional. For example, angiographic catheters are employed to inject contrast media into a vessel in order to clearly view images of the state and shape, topography, functionality and other characteristics of the vessel for diagnosing anatomical abnormalities. The catheterization thus aids in the visualization of any narrowing, blockages, or other abnormalities in the coronary arteries. Since contrast media, for example, dyes, need to be used with multiple catheters even on the same patient, there is wastage of the media. Use of multiple catheters also creates certain difficulties when used in vein grafts.

Moreover, a medical establishment needs to purchase, stock, and maintain a ready supply of catheters of various sizes and shapes for various medical specialties and services. This large inventory increases the overhead cost of the medical establishment and consequently increases the cost of medical procedures provided by the medical establishment. Furthermore, since catheters are also used to deliver wires, balloons, stents and medication to the coronary arteries, called guide catheters herein, this further raises the overall inventory of catheters in a medical establishment. Hence, there is a need for a single catheter assembly that can be used in multiple medical procedures on multiple variable anatomies. Furthermore, there is a need for a catheter assembly and a method that is more convenient to use and is easily adaptable to multiple cases, thus providing not only usage flexibility but also saving time.

Hence, there is a need for a catheter assembly configurable to multiple shapes to fit multiple anatomies. There is also a need for a single catheter assembly that decreases the time necessary for conventional medical procedures, reduces catheter cost, and provides benefits to not only the patient and the medical practitioner but also to the medical establishment, for example, by minimizing wastage of contrast media and radiation employed in a diagnostic procedure.

### SUMMARY OF THE INVENTION

This summary is provided to introduce a selection of concepts in a simplified form that are further described in the detailed description of the invention. This summary is not intended to identify key or essential inventive concepts of the claimed subject matter, nor is it intended for determining the scope of the claimed subject matter.

The catheter assembly disclosed herein addresses the above stated needs for a single catheter assembly that can be modified to obtain different shapes and angles to fit multiple anatomies.

The catheter assembly disclosed herein is configurable to multiple shapes and comprises a detachable, interchangeable and configurable outer catheter herein referred to as an “outer catheter” and a detachable, interchangeable and flexible inner catheter herein referred to as an “inner catheter”. The outer catheter comprises a first frontal end and a first distal end. The shape of the outer catheter is configured to facilitate guiding of the inner catheter into an inner cavity of a patient's anatomy. The inner catheter comprises a second frontal end and a second flexible distal end. The inner catheter is generally coaxial with the outer catheter. The second flexible distal end of the inner catheter enables the inner catheter to engage the inner cavity of the patient's anatomy. A locking element is provided for securing the inner catheter to the outer catheter when the second flexible distal end of the inner catheter engages the inner cavity of the patient's anatomy to allow equipment or material to be introduced into the inner cavity.

The catheter assembly can be configured and manipulated to different shapes and angles to allow the inner catheter to be inserted into inner cavities for multiple anatomies. For example, the catheter assembly can be configured for inserting into a patient's vascular system, the coronary arteries, etc. for diverse medical specialties and disciplines to allow diagnostic or therapeutic procedures to be performed.

### BRIEF DESCRIPTION OF THE DRAWINGS

The foregoing summary, as well as the following detailed description of the invention, is better understood when read in conjunction with the appended drawings. For the purpose of illustrating the invention, exemplary constructions of the invention are shown in the drawings. However, the invention is not limited to the specific components and methods disclosed herein.

FIG. 1A exemplarily illustrates a perspective view of a catheter assembly configurable to multiple shapes.

FIG. 1B exemplarily illustrates an orthogonal view of a catheter assembly configurable to multiple shapes.

FIG. 2A exemplarily illustrates a perspective view of an embodiment of an outer catheter of the catheter assembly.

FIG. 2B exemplarily illustrates a perspective view of another embodiment of the outer catheter of the catheter assembly.

FIG. 3 exemplarily illustrates an inner catheter of the catheter assembly.

FIG. 4 illustrates a method of performing a medical procedure on a patient by a medical practitioner using a catheter assembly.

### DETAILED DESCRIPTION OF THE INVENTION

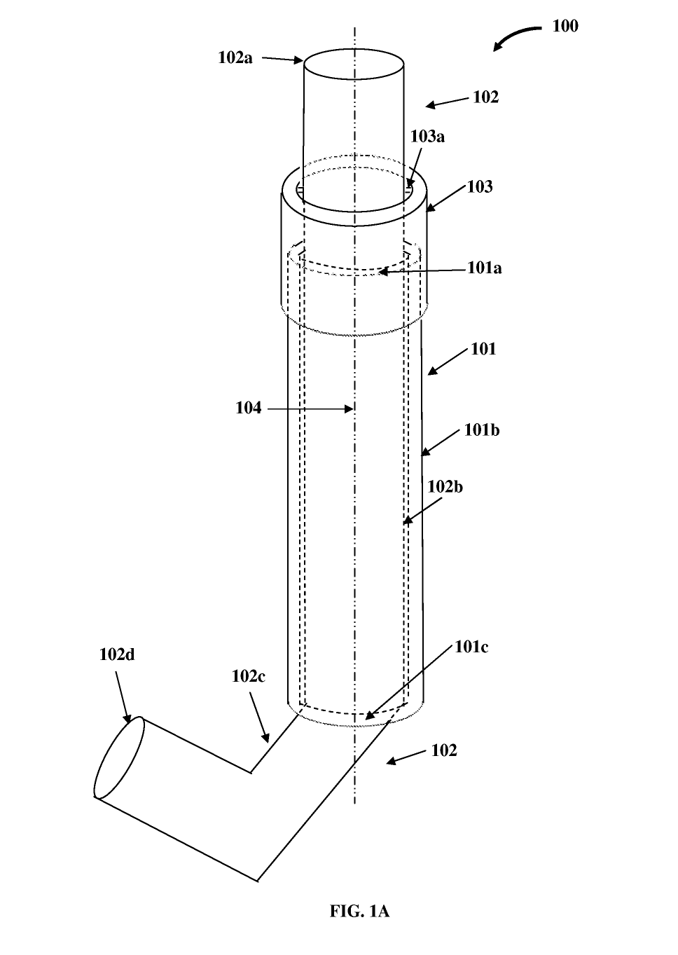

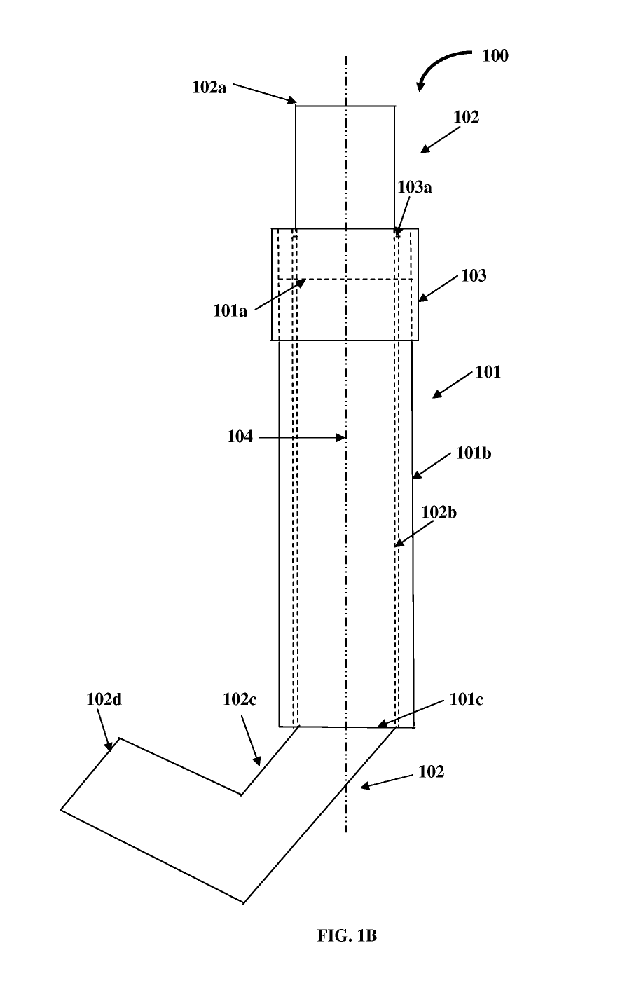

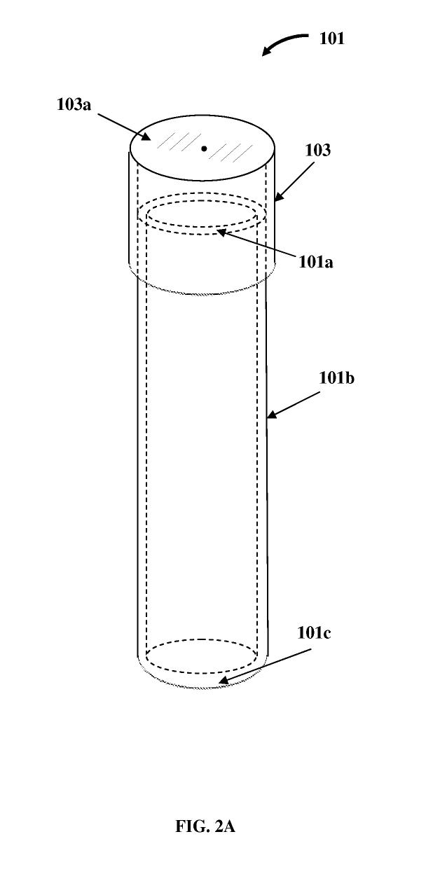

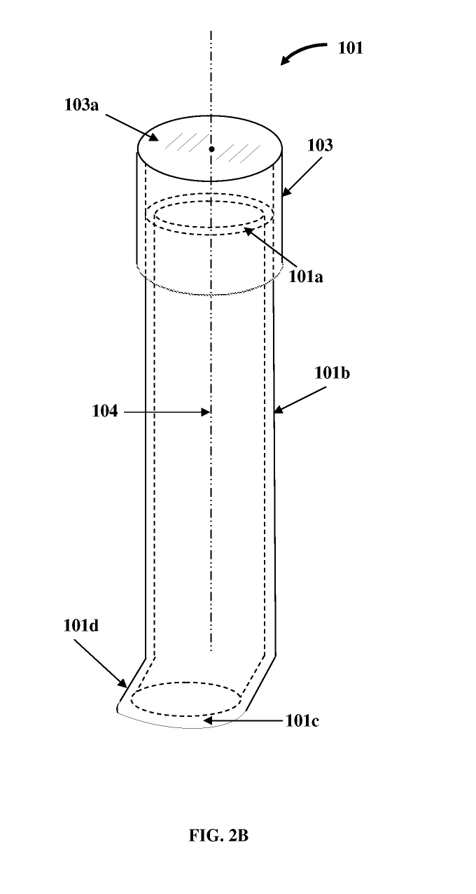

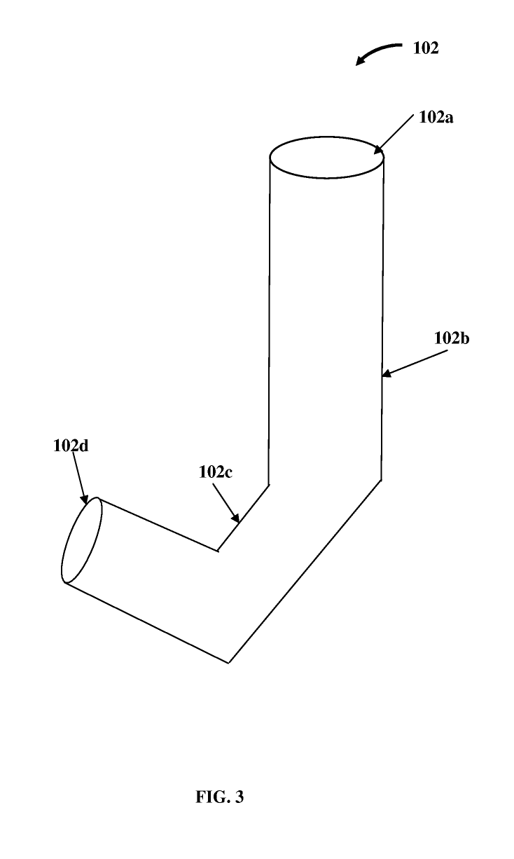

FIG. 1A exemplarily illustrates a perspective view of a catheter assembly 100 configurable to multiple shapes. An orthogonal view of the catheter assembly 100 is exemplarily illustrated in FIG. 1B. The catheter assembly 100 disclosed herein comprises a detachable, interchangeable and shape configurable outer catheter 101 as disclosed in the detailed description of FIGS. 2A-2B and a detachable, interchangeable and flexible inner catheter 102 generally coaxial with the outer catheter 101 as disclosed in the detailed description of FIG. 3. The detachable, interchangeable and shape configurable outer catheter 101 is herein referred to as an “outer catheter”. The detachable, interchangeable and flexible coaxial inner catheter 102 is herein referred to as an “inner catheter”. The thickness and material specifications of the outer catheter 101 and the inner catheter 102 are suitably adapted to fit diagnostic catheterization standards and requirements and to fit the interventional standard of a guide catheter.

The outer catheter 101 comprises a first frontal end 101a and a first distal end 101c. The shape of the outer catheter 101 is configured to facilitate guiding of the inner catheter 102 into an inner cavity of an anatomy of a patient. As used herein, the patient's anatomy refers to one of the many anatomical systems of the patient's body. The inner catheter 102 comprises a second frontal end 102a and a second flexible distal end 102d. The inner catheter 102 is slightly narrower than the outer catheter 101 to allow for the insertion of the inner catheter 102 into the outer catheter 101. The inner catheter 102 forms a force fit inside the outer catheter 101. As used herein, the term “force fit” refers to a minimal clearance between the inner catheter 102 and the outer catheter 101, so as to form a close fitting inner catheter-outer catheter assembly 100. The force fit between the coaxial inner catheter 102 and the outer catheter 101 enables relative movement between the outer catheter 101 and the inner catheter 102 when the inner catheter 102 is pushed or pulled from the second frontal end 102a or the second flexible distal end 102d with the outer catheter 101 held in an immovable position. The outer catheter 101 acts as a guiding conduit for guiding the inner catheter 102 more accurately into an inner cavity of a patient's anatomy. The second flexible distal end 102d of the inner catheter 102 is inserted into the outer catheter 101 from the first frontal end 101a of the outer catheter 101 through the first distal end 101c of the outer catheter 101 such that the second flexible distal end 102d of the inner catheter 102 extends beyond the first distal end 101c of the outer catheter 101 and is accurately guided into the inner cavity of the patient's anatomy. The outer catheter 101 and the inner catheter 102 of the catheter assembly 100 can be configured and manipulated into different shapes and angles to fit multiple anatomies and multiple inner cavities of the anatomies for enabling a medical practitioner to perform multiple medical procedures for multiple medical specialties and disciplines.

The outer catheter 101 and the inner catheter 102 are made of a biocompatible flexible polymer. The outer catheter 101 and the inner catheter 102 of the catheter assembly 100 is one of multiple shapes and sizes with one of multiple diameters and lengths. The inner catheter 102 and the outer catheter 101 are constructed using biocompatible elastomeric polymers that are durable, flexible, soft, and easily conformable to the shape of the area to be catheterized in the patient and thus minimize risk of harm to walls of the vasculature. In an embodiment, the material of construction of the outer catheter 101 is a rigid but deformable material, where the outer catheter 101 retains the shape to which the outer catheter 101 is deformed either manually or by a machine, until pressure is applied again on the outer catheter 101 to reconfigure the outer catheter 101 to a different shape. In another embodiment, the material of construction of the outer catheter 101 is pre-molded or preconfigured to facilitate its use with the coaxial inner catheter 102 in a medical procedure. The material of construction of the inner catheter 102 with respect to the outer catheter 101 is relatively flexible and deformable so as to allow the coaxial inner catheter 102 to be moved and manipulated inside and through the outer catheter 101. In an embodiment, the inner catheter 102, or a section of the inner catheter 102, for example, the second flexible distal end 102d of the inner catheter 102 is made of a deformable material that retains the shape to which the second flexible distal end 102d of the inner catheter 102 is deformed until a subsequent pressure is applied to the inner catheter 102 to reconfigure the inner catheter 102 to a different shape.

The biocompatible elastomeric polymers used in the construction of the outer catheter 101 and the inner catheter 102 comprise, for example, plastics such as urethane, polyamide such as nylon, silicone, polypropylene, polysulfone, polystyrene, polycarbonate, synthetic rubber, etc. Additionally, homopolymers and copolymers of vinyl acetate such as ethylene vinyl acetate copolymer, polyvinylchlorides, homopolymers and copolymers of acrylates such as polymethylmethacrylate, polyethylmethacrylate, polymethacrylate, ethylene glycol dimethacrylate and other similar compounds are also used. Further, to enhance lubricity in order to facilitate tracking through the vasculature, polytetraflouroethylene, polyethylene including urethane may be used.

FIG. 2A exemplarily illustrates a perspective view of an embodiment of the outer catheter 101 of the catheter assembly 100. In this embodiment, the outer catheter 101 is a generally cylindrical elongated tube 101b. The outer catheter 101 allows the inner catheter 102 to be inserted and guided through the outer catheter 101 to facilitate accurate positioning and engagement of the second flexible distal end 102d of the inner catheter 102 within an inner cavity of the patient's anatomy.

FIG. 2B exemplarily illustrates a perspective view of another embodiment of the outer catheter 101 of the catheter assembly 100. In this embodiment, the outer catheter 101 comprises a first generally cylindrical elongated tube 101b and a second section 101d angled at a point along the longitudinal axis 104 of the first generally cylindrical elongated tube 101b. The second angled section 101d of the outer catheter 101 facilitates guiding of the inner catheter 102 into the inner cavity of the patient's anatomy.

FIG. 3 exemplarily illustrates the inner catheter 102 of the catheter assembly 100. The inner catheter 102 is generally coaxial with the outer catheter 101 when the inner catheter 102 is inserted into the outer catheter 101. The inner catheter 102 of the catheter assembly 100 is one of different shapes and sizes with one of different diameters and lengths. The second flexible distal end 102d of the inner catheter 102 is one of different shapes and sizes. The second flexible distal end 102d of the inner catheter 102 is made of a soft, biocompatible, and deformable polymer for enhancing flexibility of the inner catheter 102 to facilitate easier insertion and navigation through the outer catheter 101 and also through the opening or cavity in the patient's anatomy. The second flexible distal end 102d of the inner catheter 102 is inserted into the outer catheter 101 from the first frontal end 101a of the outer catheter 101, through the generally cylindrical elongated tube 101b of the outer catheter 101 and out the first distal end 101c of the outer catheter 101 for enabling the inner catheter 102 to engage the inner cavity of the patient's anatomy.

The inner catheter 102 comprises a first generally cylindrical elongated tube 102b and a second flexible section 102c that is configured and shaped to allow the inner catheter 102 to navigate to the inner cavity in the patient's anatomy as exemplarily illustrated in FIG. 3. As used herein, the second flexible section 102c refers to a section of the inner catheter 102 that can be bent into different shapes to facilitate accurate guiding of the second flexible distal end 102d of the inner catheter 102 into the inner cavity of a patient's anatomy. The second flexible distal end 102d of the second flexible section 102c is configurable to engage with the inner cavity of the patient. The second flexible section 102c of the inserted inner catheter 102 extends beyond the first distal end 101c of the outer catheter 101 when the inner catheter 102 is inserted into the outer catheter 101 and guided into the inner cavity of the patient's anatomy.

The inserted inner catheter 102 accommodates one or more surgical instruments, for example, stents and wires, and materials, for example, dyes that are inserted into the inner catheter 102 through the second frontal end 102a. In an embodiment, the opening at the second flexible distal end 102d of the inner catheter 102 is made of a deformable material to allow the shape of the second flexible distal end 102d of the inner catheter 102 to be configured or adjusted to accommodate stents, wires, dyes and the like and/or for insertion of such materials into the desired inner cavity in the anatomy of the patient's body. In an embodiment, the inner catheter 102 is made of a less rigid material than the outer catheter 101, and can be configured and manipulated into multiple angles and be rotated up to 360 degrees and be moved inwards and outwards inside the outer catheter 101.

The inner catheter 102 is secured to the outer catheter 101 using a locking element 103, for example, a locking diaphragm 103a, a clamp-type band, an adapter, etc. when the second flexible distal end 102d of the inner catheter 102 engages the inner cavity in the anatomy where the second flexible distal end 102d is to be inserted. The locking element 103 is a clamp-type band that is provided at the first frontal end 101a of the outer catheter 101 and attached around the inner catheter 102 to secure the inserted inner catheter 102 to the outer catheter 101. When secured, there is no relative movement of the inner catheter 102 with respect to the outer catheter 101 about the locking element 103. When the inner catheter 102 of the inner catheter-outer catheter assembly 100 reaches the desired location of the patient's inner cavity, the locking element 103 is tightened around the catheter assembly 100 to secure the inserted inner catheter 102 to the outer catheter 101.

In an embodiment, the locking element 103 comprises, for example, a locking diaphragm 103a as illustrated in FIGS. 1A-1B and FIGS. 2A-2B. The locking diaphragm 103a is provided at the first frontal end 101a of the outer catheter 101 for securing the inserted inner catheter 102 to the outer catheter 101. The locking diaphragm 103a is made of an elastic material that secures the inner catheter 102 to the outer catheter 101.

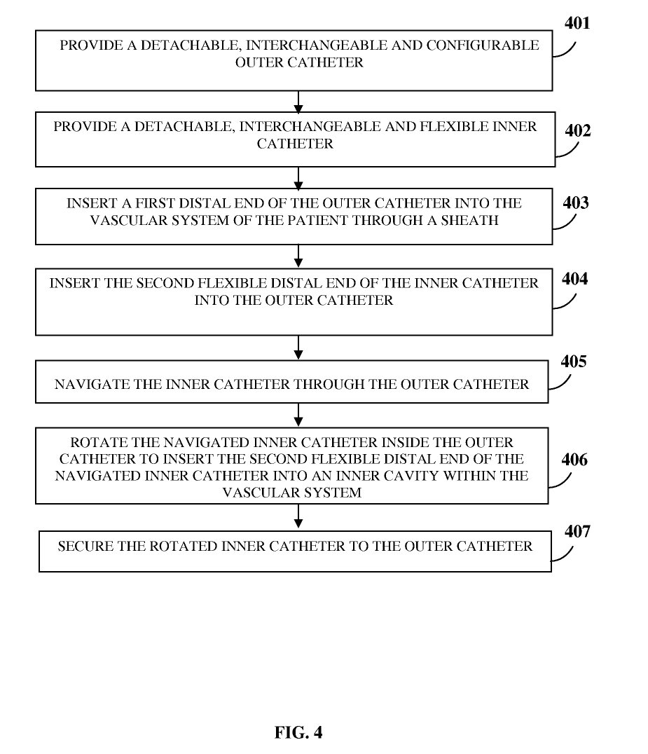

FIG. 4 illustrates a method of performing a medical procedure on a patient by a medical practitioner using a catheter assembly 100. An outer catheter 101 of one of multiple shapes and sizes with one of multiple diameters and lengths as disclosed in the detailed description of FIGS. 2A-2B is provided 401. The outer catheter 101 comprises a first frontal end 101a and a first distal end 101c. An inner catheter 102 of one of multiple shapes and sizes with one of multiple diameters and lengths as disclosed in the detailed description of FIG. 3 is provided 402. The inner catheter 102 comprises a second frontal end 102a and a second flexible distal end 102d. The inner catheter 102 is generally coaxial with the outer catheter 101. The inner catheter 102 further comprises a first generally cylindrical elongated tube 102b and a second flexible section 102c angled at a point with respect to the longitudinal axis 104 of the outer catheter 101 and thereafter angled again at one or more points along the axis of the inner catheter 102 as required for the medical procedure.

The second flexible section 102c of the inserted inner catheter 102 is made of a soft, biocompatible and deformable polymer and conforms to the shape and form of an inner cavity of the patient. A vascular system of a patient is accessed using a needle (not shown) and a small fixed plastic tube (not shown), herein referred to as a “sheath”, is inserted into the vascular system of the patient. As used herein, the sheath refers to a fixed plastic tube used routinely in angiographic procedures. The first distal end 101c of the outer catheter 101 is inserted 403 into the vascular system of the patient's anatomy through the sheath.

The second flexible distal end 102d of the inner catheter 102 is inserted 404 into the outer catheter 101 from the first frontal end 101a of the outer catheter 101 to create a single catheter assembly 100. The inner catheter 102 is navigated 405 through the outer catheter 101 till the second flexible distal end 102d of the navigated inner catheter 102 emerges from the first distal end 101c of the outer catheter 101 and reaches a predetermined location inside the patient's anatomy. The second flexible section 102c of the inserted inner catheter 102 extends beyond the first distal end 101c of the outer catheter 101. The navigated inner catheter 102 is rotated 406 inside the outer catheter 101 to insert the second flexible distal end 102d of the navigated inner catheter 102 into the inner cavity within the vascular system at the predetermined location. The rotated inner catheter 102 is then secured 407 to the outer catheter 101 by a locking element 103 as exemplarily illustrated in FIGS. 1A-1B and FIGS. 2A-2B. One or more surgical instruments and materials are inserted through the second frontal end 102a of the secured inner catheter 102, to enable the medical practitioner to perform the medical procedure on the anatomy of the patient using the catheter assembly 100.

The catheter assembly 100 is configured and manipulated into different shapes and angles to fit multiple anatomies and multiple inner cavities of the anatomy for enabling the medical practitioner to perform multiple medical procedures, for example, cardiac surgery or surgery on any vascular system under diverse medical specialties and disciplines to perform diagnostic or therapeutic procedures.

Consider an example of performing a medical procedure using the catheter assembly 100. An outer catheter 101 as exemplarily illustrated in FIG. 2A is provided. An inner catheter 102 as exemplarily illustrated in FIG. 3 is provided. The medical practitioner accesses the vascular system of a patient using a needle. The medical practitioner then inserts the sheath through the access into the vascular system of the patient. The medical practitioner then inserts the first distal end 101c of the outer catheter 101 through the sheath over a flexible wire, for example, a guide wire (not shown). As used herein, the guide wire refers to a wire that is routinely used in angiographic procedures and removed after the desired location of the inner catheter 102 is achieved. The medical practitioner then inserts the second flexible distal end 102d of the inner catheter 102 into the outer catheter 101 from the first frontal end 101a of the outer catheter 101. The medical practitioner navigates the inserted inner catheter 102 through the outer catheter 101 till the second flexible distal end 102d of the navigated inner catheter 102 emerges from the first distal end 101c of the outer catheter 101 and reaches the desired location in the patient's body, for example, the root of the aorta of the patient. The navigated inner catheter 102 is rotated inside the inserted outer catheter 101 to enable insertion of the second flexible distal end 102d of the navigated inner catheter 102 into the inner cavity of the aorta of the patient. At this point the guide wire is removed. The rotated inner catheter 102 is secured to the outer catheter 101 by the locking element 103 comprising, for example, the locking diaphragm 103a. The rotated inner catheter 102 engages the origins of the left main and right coronary arteries. The medical practitioner then inserts one or more cardiac surgical instruments and materials through the second frontal end 102a of the secured inner catheter 102 to perform the cardiac procedure on the patient. The catheter assembly 100 is used for performing similar procedures, for example, angiography and intervention of peripheral arteries, for example, lower extremity arteries, carotid arteries, renal arteries or other blood vessels.

The catheter assembly 100 and the method for performing a medical procedure using the catheter assembly 100 disclosed herein enables the movement and adjustment of the inner catheter 102 at the point where the medical procedure is performed for different anatomies, for example, coronary arteries that are of different shapes and forms due to genetic and individual variations, in cases of tortuosity of the aorta and of saphenous vein grafts, resulting in substantial saving of time and cost. In diagnostic procedures, the catheter assembly 100 disclosed herein reduces the amount of contrast media used, thereby reducing wastage of the media.

The catheter assembly 100 allows a single catheter assembly to be used for multiple procedures by changing the shape of the outer catheter 101 or the opening of the inner catheter 102. The catheter assembly 100 also provides cost savings by way of lower inventory. The catheter assembly 100 further reduces the amount of contrast media used and also reduces the radiation employed, by reducing the exposure time required in taking an in-dwelling catheter out of the anatomy of a patient and then advancing and trying other catheter or catheters.

The foregoing examples have been provided merely for the purpose of explanation and in no way are to be construed as limiting of the present invention. While the invention has been described with reference to various embodiments, it is understood that the words, which have been used herein, are words of description and illustration, rather than words of limitation. Additionally, although the invention has been described herein with reference to particular means, materials and embodiments, the invention is not intended to be limited to the particulars disclosed herein; rather, the invention extends to all functionally equivalent structures, methods and uses, such as are within the scope of the appended claims. It will be appreciated by those skilled in the art, having the benefit of the teachings of this specification, that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I claim:

1. A catheter assembly configurable to multiple shapes, comprising:
a detachable, interchangeable and configurable outer catheter comprising a first frontal end and a first distal end, wherein shape of said outer catheter is configured to facilitate guiding of a detachable, interchangeable and flexible inner catheter into an inner cavity of an anatomy of a patient;
said detachable, interchangeable and flexible inner catheter comprising a second frontal end and a second flexible distal end, said inner catheter generally coaxial with said outer catheter, wherein said second flexible distal end of said inner catheter enables said inner catheter to engage said inner cavity of said anatomy of said patient; and
a locking element for securing said inner catheter to said outer catheter when said second flexible distal end of said inner catheter engages said inner cavity of said anatomy of said patient to allow one of equipment and material to be introduced into said inner cavity;
whereby said catheter assembly is configured and manipulated to fit multiple anatomies and multiple inner cavities of said multiple anatomies for enabling a medical practitioner to perform a plurality of medical procedures for a plurality of medical specialties and disciplines.

2. The catheter assembly of claim 1, wherein said outer catheter is a generally cylindrical elongated tube.

3. The catheter assembly of claim 1, wherein said outer catheter comprises a first generally cylindrical elongated tube and a second section angled at a point along the longitudinal axis of said first generally cylindrical elongated tube, wherein said second angled section of said outer catheter facilitates guiding of said inner catheter into said inner cavity of said anatomy of said patient.

4. The catheter assembly of claim 1, wherein said outer catheter is one of a plurality of shapes and sizes with one of a plurality of diameters and lengths.

5. The catheter assembly of claim 1, wherein said inner catheter comprises a first generally cylindrical elongated tube and a second flexible section, wherein said second flexible distal end of said second flexible section is configurable to engage with said inner cavity of said patient, wherein said second flexible section of said inner catheter extends beyond said first distal end of said outer catheter.

6. The catheter assembly of claim 1, wherein said inner catheter is one of a plurality of shapes and sizes with one of a plurality of diameters and lengths.

7. The catheter assembly of claim 1, wherein said inner catheter accommodates one or more of a plurality of surgical instruments and materials inserted through said second frontal end of said inner catheter.

8. The catheter assembly of claim 1, wherein said second flexible distal end of said inner catheter is one of a plurality of shapes and sizes.

9. The catheter assembly of claim 1, wherein said locking element comprises a locking diaphragm provided at said first frontal end of said outer catheter for securing said inner catheter to said outer catheter.

10. The catheter assembly of claim 1, wherein said outer catheter and said inner catheter are made of a biocompatible flexible polymer.

11. The catheter assembly of claim 1, wherein said second flexible distal end of said inner catheter is made of a soft, biocompatible, and deformable polymer.

12. A method of performing a medical procedure on a patient by a medical practitioner using a catheter assembly, comprising:
providing said catheter assembly comprising:
a detachable, interchangeable and configurable outer catheter of one of a plurality of shapes and sizes with one of a plurality of diameters and lengths, comprising a first frontal end and a first distal end, wherein shape of said outer catheter is configured to facilitate guiding of a detachable, interchangeable and flexible inner catheter into an inner cavity of an anatomy of a patient;
said detachable, interchangeable and flexible inner catheter of one of a plurality of shapes and sizes with one of a plurality of diameters and lengths, comprising a second frontal end and a second flexible distal end, said inner catheter generally coaxial with said outer catheter, wherein said second flexible distal end of said inner catheter enables said inner catheter to engage said inner cavity of said anatomy of said patient; and
a locking element for securing said inner catheter to said outer catheter when said second flexible distal end of said inner catheter engages said inner cavity of said anatomy of said patient to allow one of equipment and material to be introduced into said inner cavity;
inserting said first distal end of said outer catheter into a vascular system of said anatomy of said patient through a sheath inserted into said vascular system using a needle;
inserting said second flexible distal end of said inner catheter into said outer catheter from said first frontal end of said outer catheter;
navigating said inner catheter through said outer catheter till said second flexible distal end of said navigated inner catheter emerges from said first distal end of said outer catheter and reaches a predetermined location;
rotating said navigated inner catheter inside said outer catheter to insert said second flexible distal end of said navigated inner catheter into an inner cavity within said vascular system at said predetermined location; and
securing said rotated inner catheter to said outer catheter by said locking element;
whereby said catheter assembly configured and manipulated to fit multiple anatomies and multiple inner cavities of said multiple anatomies enables said medical practitioner to perform said medical procedure on said anatomy of said patient.

13. The method of claim 12, wherein said outer catheter comprises a first generally cylindrical elongated tube and a second section angled at a point along the longitudinal axis of said first generally cylindrical elongated tube, wherein said second angled section of said outer catheter facilitates guiding of said inner catheter into said inner cavity of said anatomy of said patient.

14. The method of claim 12, wherein said inner catheter comprises a first generally cylindrical elongated tube and a second flexible section, wherein said second flexible distal end of said second flexible section is configurable to engage with said inner cavity of said patient, wherein said second flexible section of said inner catheter extends beyond said first distal end of said outer catheter.

15. The method of claim 12, wherein said outer catheter and said inner catheter are made of a biocompatible flexible polymer.

16. The method of claim 12, wherein said second flexible distal end of said inner catheter is made of a soft, biocompatible and deformable polymer for conforming to shape and form of said inner cavity of said patient.
